(19) United States
(12) Patent Application Publication
(10) Pub. No.: US 2026/0069563 A1
(43) Pub. Date: Mar. 12, 2026

(54) CETOLEIC ACID COMPOSITION

(71) Applicant: Epax Norway AS, Ålesund (NO)

(72) Inventors: Iren Merete Skjåstad STOKNES, Ålesund (NO); Derek TOBIN, Kolsås (NO)

(73) Assignee: Epax Norway AS, Ålesund (NO)

(21) Appl. No.: 19/105,921

(22) PCT Filed: Aug. 24, 2023
(86) PCT No.: PCT/NO2023/060035

§ 371 Date: Feb. 24, 2025

(30) Foreign Application Priority Data
Aug. 25, 2022 (NO) 20220910

(51) Int. Cl. A61K 31/201; A61K 31/202; A61K 35/60; A61P 3/10; A61P 17/00; A61P 17/02; C12N 9/20; C12P 7/6458

(52) U.S. Cl. CPC A61K 31/201; A61K 31/202; A61K 35/60; A61P 3/10; A61P 17/00; A61P 17/02; C12N 9/20; C12P 7/6458; C12Y 301/01003

(57) ABSTRACT

The present invention relates to an enriched LCMUFA composition comprising cetoleic acid. Furthermore, the invention relates to a method for providing an enriched composition comprising cetoleic acid, and to the use of such compositions.

### FIELD OF THE INVENTION

The present invention relates to LCMUFA compositions, and particularly to enriched LCMUFA compositions comprising cetoleic acid. Furthermore, the invention relates to a method for providing compositions comprising cetoleic acid, and to different applications of the LCMUFA compositions.

### BACKGROUND OF THE INVENTION

A wealth of scientific studies supports omega-3 fatty acids' key role in improving risk factors for heart disease, reduction of inflammation, and promotion of brain health during pregnancy and early life. With such wide-ranging and proven positive effects on human health, the omega-3 ingredients market is big business. Back in the 1960s, a multinational epidemiological study, Keys et al, Acta Med Scand Suppl. 1966, 460, 1-392, led to the discovery that also long-chain monounsaturated fatty acids (LCMUFAs) derived from fish such as mackerel and herring had positive health effects, particularly in relation to coronary heart disease. This study boosted the general interest in mono-unsaturated fatty acids, and the promotion of the Mediterranean diet, rich in olive oil derived oleic acid, a mono-unsaturated fatty acid, which is still regarded as one of the healthiest diets. The human body contains many different fats, which help us store energy, keep us insulated, protect our vital organs, and provide important structural functions. Several lipids have important biological activity, meaning they elicit specific responses in the body.

The dietary fat humans consume can be separated into two categories: saturated and unsaturated. The latter is commonly seen as “healthy fat” because it helps to raise levels of good cholesterol, HDL, and reduce unhealthy triglycerides. The unsaturated fat can be classified as two main types: monounsaturated (MUFAs) and polyunsaturated fats (PUFAs). Long chain fatty acids are those with carbon chains longer than 18, C20-C22, and fish oil is rich in these lipids. The LCPUFAs include the omega-3 fatty acids EPA and DHA which are important for health but are produced in humans in small amounts. Numerous studies show health effects of EPA and DHA including reducing inflammation, lowering the risk of Alzheimer's disease, and reducing the impact of cardiovascular disease. Dietary intake of EPA and DHA is therefore important to maintain healthy levels of these fatty acids.

Fish and marine life are rich sources of EPA and DHA but in addition certain fish also contain high levels of LCMUFAs. Compared to EPA and DHA, there is limited knowledge about the health benefits of marine LCMUFAs. Cetoleic acid is an omega-11 marine lipid found in higher concentrations in North Atlantic fish, such as herring, mackerel and tobis, compared to the more EPA- and DHA-rich South American fish, such as anchovies and sardines.

LCMUFAs, including cetoleic acid, can be utilized as an energy source by mitochondrial β-oxidation and peroxisomal β-oxidation, as explained by Bremer et al, J Lipid Res, February 1982, 23(2),43-56. Adipose tissue is the main storage site of triglycerides and feeding studies with a cetoleic-rich oil showed high uptake of cetoleic acid in adipose tissue suggesting that cetoleic acid is stored in the adipose tissue and may have important effects in this tissue. Early studies, see Yang et al, Lipids Health Dis, Nov. 22, 2016, 15(1), 201, and Yang et al, Mol Nutr Food Res 2016, 60 (10), 2208-2218, have shown that fish oils rich in cetoleic acid reduce systemic inflammation and reduce the degree of atherosclerosis in mice. Other positive health effects have been noted in relation to reducing inflammation and in hindering the development of metabolic syndrome and obesity-related metabolic dysfunction.

Cell culture studies and human clinical studies support the hypothesis that fish-oil rich in cetoleic acid promote the conversion of α-linolenic acid to EPA, as disclosed by Østbye et al, BR, J Nutr. Oct. 14, 2019, 122(7), 755-768.

Studies in the late 1990s showed that cetoleic-rich oils and EPA and DHA may have different activities in the body. EPA and DHA supplementation lead to beneficial plasma lipid effects which are not seen with cetoleic-rich oils. In other words, the health benefits of these two fatty acids may cover different areas of metabolic health. Several lines of evidence support this. In vitro studies with liver cells demonstrate that EPA and DHA reduce the cholesterol and triglyceride content of liver cells, but that this reduction was greater when cells were co-administered with a cetoleic-rich oil, please see Yoshinaga et al, J Oleo Sci. May 1, 2021, 70(5), 731-736. The impact of omega-3 fatty acids is mostly on triglycerides and HDL, whereas cetoleic-rich oil affects non-HDL cholesterol and insulin sensitivity, reference is made to Yang et al, J Agric Food Chem, Jul. 13, 2011, 59(13), 7482-9. This complementary effect is of interest in providing comprehensive metabolic protection. Indeed, a combined beneficial effect has been suggested in metabolic syndrome following data from an animal model whereby EPA/DHA and cetoleic-acid rich oils show both combined and complementary activity within metabolic syndrome, with combined effects on decreased plasma non-HDL cholesterol, improved hyperinsulinemia, liver fat, and reduced plasma lipid levels, as provided by Yang et al, Lipids Health Dis, Dec. 1, 2015, 14, 155.

As for the LCPUFAs EPA and DHA, levels of the omega-11 LCMUFAs such as cetoleic acid are dependent on dietary intake as the body is unable to synthesize them.

EP2682116 of Nippon Suisan Kaisha describes an agent for use in the amelioration of metabolic syndrome, wherein the agent comprises a MUFA, but almost no LCPUFAs.

LCMUFAs, including cetoleic acid, is present in natural oils and is a valuable resource. However, the MUFAs are typically present in a mixture with a range of other fatty acids. Hence, there is a need for compositions comprising LCMUFAs, and for methods for separating the MUFAs from other fatty acids, for the provision of MUFA compositions.

### BRIEF SUMMARY OF THE INVENTION

The invention provides compositions comprising long chain monounsaturated fatty acids (LCMUFAs), particularly cetoleic acid, to methods for providing such compositions, and to the use of these.

In one aspect, the invention relates to a LCMUFA composition comprising
C22:1 n11 (cetoleic acid) and C20:5 n3 (EPA), wherein;
the concentration of C22:1 n11 is at least 16.0 wt. %; and
the EPA concentration is at least 3 wt. %.

In the compositions of the invention the DHA concentration is low. In one embodiment the DHA concentration is at maximum 4.0 wt. % of the composition. In one embodiment the EPA:DHA ratio is at least 4:1.

In one embodiment, the present invention relates to an enriched LCMUFA composition wherein the concentration of C22:1 n11 is at least 39 wt. %.

In a second aspect, the invention relates to a method for preparing a composition of LCMUFAs, the method comprising the step of:
i) mixing a natural oil with a lipase to obtain a mixture comprising LCPUFAs on glyceride form and LCMUFAs on ethyl ester form;
ii) distilling the mixture obtaining an LCMUFA composition as a first fraction and a LCPUFA composition as a second fraction.

In one embodiment, the method is for preparation of the LCMUFA composition of the first aspect. The method enables separation of LCMUFAs such as cetoleic acid and gondoic acid from EPA and DHA.

In further aspects, the invention relates to LCMUFA compositions according to the first aspect for use in therapy or in health indications, and to the use in feed or food.

### BRIEF DESCRIPTION OF THE DRAWINGS

FIG. 1 provides results from a study using a wound healing model (Example 4) showing the area of wound 14 hours after wounding and treatment of cells, cells being added a LCMUFA concentrate or DHA, respectively.

FIG. 2 shows the melanin concentration (μg melanin/μg cellular DNA) in human fibroblasts, n=4, after oxidative stress, from the wound healing model of Example 4.

FIG. 3 shows the relative expression of inflammation related genes (log 2) in human fibroblasts under oxidative stress, from the wound healing model of Example 4.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

In the present invention, “monounsaturated fatty acid” or “MUFA” is a fatty acid having one double bond, while “polyunsaturated fatty acid” or “PUFA” refers to a fatty acid having four double bonds or more. MUFAs and PUFAs with a long chain fatty acid having 20 or more carbon atoms are described as LCMUFAs or LCPUFAs. A MUFA with 20 carbons is called eicosenoic acid (icosenoic acid) in the IUPAC name system and cis-icos-9-enoic acid (n-11, idiomatic gadoleic acid), cis-icos-11-enoic acid (n-9, common name gondoic acid) and so on. A MUFA with 22 carbon atoms is called docosenoic acid in the IUPAC name system, cis-docos-11-enoic acid (n-11, common name cetoleic acid), cis-docos-13-enoic acid (n-9, common name erucic acid) and the like.

In the present invention the following nomenclature is used:
s Gondoic acid, C20:1 n9;
Gadoleic acid, C20:1 n11;
Erucic acid C22:1 n9;
Cetoleic acid, C22:1 n11;
Eicosapentaenoic acid (EPA), C20:5 n3;
Docosahexaenoic acid (DHA), C22:6 n3.

The invention is directed to compositions, and to methods for providing such compositions, wherein the compositions comprise both LCMUFAs and LCPUFAs obtained from a natural oil. The invention provides LCMUFA compositions, and particularly enriched LCMUFA compositions comprising cetoleic acid. The invention further provides a method for producing a composition comprising a high concentration of cetoleic acid from a natural oil, by isolating this from other lipids.

Enriched compositions are provided which may provide complementary benefits from EPA and cetoleic acid and other fatty acids. The applicant has examined the evidence supporting the marine derived LCMUFAs; particularly omega-11 cetoleic acid (C22:1 n-11), and also omega-9 gondoic acid (C20:1 n-9), and provides new compositions, for example for the marine lipid ingredients market, or for human health.

Thus, in one aspect, the invention provides an LCMUFA composition comprising
C22:1 n11 (cetoleic acid) and C20:5 n3 (EPA) wherein;
the concentration of C22:1 n11 is at least 16 wt. %;
the EPA concentration is at least 3 wt. %.

In the compositions of the invention the DHA concentration is low, and hence the ratio of either of EPA:DHA and cetoleic acid:DHA, is high. In one embodiment, the DHA concentration is maximum 4 wt. % of the composition.

The MUFA composition of the invention, comprising a high concentration of cetoleic acid, is derived from a natural starting oil comprising at least both cetoleic acid and EPA. The starting oil, also called raw material, either being a crude oil or a refined oil used as starting oil for provision of the composition of the invention, typically comprises a least 9 wt. % cetoleic acid, and at least 3 wt. % EPA, more preferably at least 15 wt. % cetoleic acid and at least 5 wt. % EPA, such as at least 7 wt. % EPA. The applicant has found that it is possible to obtain compositions comprising both health-valuable LCMUFAs and LCPUFAs from the same starting oil, in commercially useful amounts. Herein is disclosed a process for isolating separate fractions of certain LCMUFAs from certain LCPUFAs. The invention hence also relates to a method of producing compositions comprising a high concentration of LCMUFAs, particularly of cetoleic acid from natural oils, and in addition, such compositions are disclosed and claimed. Methods have been identified that enable separation of the MUFA C22:1 n11 (cetoleic acid), from other fatty acids, including from the polyunsaturated fatty acid with same length, C22:6 n3 (DHA), as further shown below.

The raw material, used for provision of the composition of the invention is a natural oil from a marine source, such as from fish oil, squid oil, krill oil or algal oil, and preferably from fish oil. Particularly the raw material is a North Atlantic fish oil, such as oil from herring, mackerel, capelin, cod, saithe or tobis. The raw material may also come from other fish species comprising cetoleic acid, such as e.g. pollock or saury being North Pacific fish species. Most preferably, the raw material is oil from herring or mackerel, and most preferably from herring. These North Atlantic oils are favourable due to the higher volume available, because of the high concentration of cetoleic acid, and due to the EPA:DHA ratio in these. Furthermore, the North Atlantic fish oils are rich in gondoic acid, c20:1 n9, being a further reason for using such as oils for provision of the composition of the invention. Hence, in one embodiment, the provided MUFA composition of the invention is a fish oil MUFA composition. Furthermore, in one embodiment, the fatty acids of the starting oil for preparation of the compositions are on the triglyceride form. The provision of the MUFA composition of the invention accordingly also includes a valuable use of a resource where parts of the material have so far been little used. It is particularly beneficial to be able to preserve and utilize both MUFAs and PUFAs from oils high in MUFAs. As is today, the North Atlantic oils have been regarded as a second choice, typically for EPA and DHA production as the level of these are lower than in oils like anchovy and sardines. Rather than using typical EPA/DHA sources such as anchovy oil, or using MUFA-rich oils for EP/DHA production but not utilizing the MUFA component, the invention provides a method wherein oils with a high MUFA content is utilized, providing valuable MUFA compositions, and also recovering and utilizing the PUFAs.

In some embodiments, the composition of the invention comprises 16.0-60.0 wt % cetoleic acid, such as 18.0-50.0 wt % cetoleic acid, more preferably 20.0-50.0 wt % cetoleic acid, even more preferably 39.0-50.0 wt % cetoleic acid, and preferably about 40-45 wt. % cetoleic acid. In one embodiment, the concentration of cetoleic acid is at least 39 wt. %. The enrichment of cetolelic acid can for example be achieved as disclosed below.

In some embodiments, the composition comprises LCMUFAs in an amount of at least 20.0 wt. %, such as at least 30.0%. More preferably, an enriched composition of the invention comprises LCMUFAs in an amount of at least 47 wt. %, preferably at least 50 wt. %, such as at least 60 wt. %. In one embodiment, the composition comprises LCMUFAs in a range of 47-80 wt. %, such as 50-76 wt. %, and preferably in the range of 60-65 wt. %. The LCMUFAs comprise the C20 fatty acid gondoic acid and may further comprise gadoleic acid and the C22:1 fatty acid erucic acid, in addition to cetoleic acid. The provided composition is hence enriched in LCMUFAs, i.e. C20:1 and 22:1, and the concentration of LCMUFAs is considerably higher in the claimed composition, than in the starting oil used for preparation. However, the ratio of C22:1 to C20:1 is similar to that found in the starting oil (natural oil). In one embodiment, the weight ratio between C22:1 and C20:1 is from 1.5:1 to 2.6:1, such as about 1.75:1. More particularly, the weight ratio of C22:1 n11:C20:1 n9 is about 1.75:1. In one embodiment, the weight ratio of C22:1 n11:C20:1 n9 is from 1.5:1 to 2.6:1.

In some embodiments, the concentration of gondoic acid, C20:1 n9, is in the range of 9-28 wt. %, such as 15-28 wt. %, e.g. 18-28 wt. %. More preferably the concentration of C20:1 n9 is in the range of 18-26 wt. %. Such high concentration of gondoic acid, in combination with a high cetoleic acid concentration, is particularly obtainable from the North Atlantic fish oils, unlike from typical Pacific fish specifies like pollock and saury. Oils from Pacific fish species typically comprise more gadoleic acid, C20:1 n11, rather than gondoic acid (C20:1 n9). The enriched compositions and high concentrations are obtainable by the method disclosed below, particularly when the method comprises more than one concentration round.

In some embodiments, the amount of gadoleic acid, C20:1 n11, is particularly low in the compositions of the invention. This is maximum 2.0 wt. %, preferably 1.0-2.0 wt. %.

In some embodiments, the amount of erucic acid, C22:1 n9, is particularly low in the compositions of the invention. This is max 3.0 wt. %, preferably 2.0-3.0 wt. %.

The EPA concentration in the composition is at least 3.0 wt %, and preferably at least 5.0 wt. %. In some embodiments, the concentration is 5.0-11.0 wt. %. In some embodiments, the EPA concentration is about the same as in the starting oil (raw oil), or slightly lower. In exemplary embodiments, e.g. using mackerel or herring oil as starting oil, having an EPA concentration of about 8%, the composition of the invention comprises about 5-8% EPA. In another embodiment, the EPA concentration is even higher, such as up to 40 wt. % of the composition. Hence, the invention provides a composition comprising an enriched amount of cetoleic acids, as disclosed, and an EPA concentration of 5-40 wt %, such as 20-40 wt %. To obtain an EPA enriched MUFA composition, an EPA concentrate may be added to a MUFA composition of the invention.

Hence, in one embodiment, an EPA-enriched LCMUFA composition of the invention comprises C22:1 n11 (cetoleic acid) and C20:5 n3 (EPA), wherein;
the concentration of C22:1 n11 is at least 16.0 wt. %, such as 16-60 wt. %, e.g. 30-50% wt. % of the composition;
the EPA concentration is in the range of 20-40 wt. % of the composition, such as 20-30 wt. % of the composition.

In one embodiment, DHA may be present in a concentration at maximum 4.0 wt. % of the composition. Furthermore, the EPA-enriched MUFA composition preferably further comprises gondoic acid, C20:1 n9, in a concentration of 9-28 wt. % of the composition.

In preferred embodiments, the composition of the invention comprises only a low amount of DHA. The applicant has surprisingly been able to separate LCMUFAs, particularly C22:1 MUFAs, i.e. cetoleic acid and erucic acid, from this polyunsaturated C22 fatty acid. Hence, the provided MUFA composition of the invention is significantly lower in DHA than the starting oil, and accordingly has a high ratio of EPA:DHA. The MUFA composition of the invention typically has an about 10-fold higher ratio of cetoleic acid:DHA, compared to the natural starting oil. Crude oil of mackerel typically comprises about 11-12 wt. % DHA, while herring oil typically comprises about 9-10 wt. % DHA. However, the MUFA composition of the invention comprises very little DHA, but has a high concentration of cetoleic acid, the fatty acid having the same length. In one embodiment, the LCMUFA composition of the invention has a ratio of cetoleic:DHA of at least 10:1, such as at least 15:1, more preferably at least 20:1.

Furthermore, the MUFA composition of the invention has a high ratio of EPA:DHA. In one embodiment, the LCMUFA composition has a ratio of EPA:DHA of 4.0:1-16.0:1, more preferably 4.0:1-8.0:1. In compositions of the invention wherein the amount of EPA is high, such as in EP-enriched MUFA compositions, the DHA content is anyhow low and the EPA:DHA ratio may be as high as 50:1. Hence, in one embodiment the EPA:DHA ratio is from 4:1 to 50:1. Hence, the concentration of EPA is minimally at least four times higher than the concentration of DHA in the MUFA composition. However, the more the composition is enriched with cetoleic acid, e.g. by concentration by distillation, this ratio decreases.

The concentration of DHA is accordingly surprisingly low in the provided MUFA composition. In some embodiments, the DHA concentration of the MUFA composition is at maximum 4.0 wt. %, such as being in the range of 0.5-3.5 wt. %, which i.e. is considerably lower than in suitable marine raw oils. Compositions wherein no DHA is present, i.e. compositions with a concentration of 0 wt % DHA, fall within the claimed compositions, i.e. the composition has a DHA concentration of 0.0-4.0 wt %. A low amount of DHA may be desirable in compositions, for example, for cardiovascular health such as for treatment of heart disorders to avoid negative impact on blood parameters as TG, LDL, HDL and total cholesterol. Furthermore, as the compositions of the invention comprise a high concentration of MUFAs and very little of the DHA PUFA, the fatty acids of the compositions of the invention are less prone to oxidize than composition with a higher concentration of PUFAs. The compositions of the invention hence have a pleasant taste and odour and may be included in food.

Further reasons for providing the claimed composition, is that the applicant wants to provide a product with cetoleic acid and little DHA and EPA to give customers choices, so they can manage their lipid intake. Customers are likely to already be taking an omega-3 supplement and from a commercial perspective it is not wanted to replace conventional high concentrate omega-3 which have a dominant place in the fish oil market. In addition, alternative oils such as a cetoleic rich oil, will never supply the levels of EPA and DHA sold in omega-3 concentrates. That leaves the dilemma that customers may be taking one capsule for omega-3 and one for cetoleic acid, but with the danger of doubling their EPA and DHA intake which is regulated by the FDA with a maximum EPA and DHA intake of 3 g. Customers therefore risk taking too much omega-3 when taking additional cetoleic acid supplements. A low EPA-DHA Cetoleic acid supplement is therefore required. Furthermore, EPA and DHA are known to reduce plasma level of triglycerides (TG) and have minor changes in total cholesterol. EPA elicits a slight decrease in the level of total cholesterol whereas DHA exhibits a slight elevation mostly due to the increase in HDL cholesterol. As demonstrated by Sarbolouki et al, 2013, Singapore Med J., 54(7):387-90, EPA supplementation could improve insulin sensitivity by decreases in fasting plasma glucose, HbA1c and homeostasis model assessment of insulin resistance. Accordingly, EPA exhibits a negative correlation with plasma levels of TG and insulin. However, EPA exhibits only marginal to no effect on total cholesterol. The main claim for LCMUFA cardiovascular protection comes from its ability to lower LDL levels without decreasing HDL levels. Long-term supplementation of LCMUFA (Yang, 2015) indicated that LCMUFA had marginal to no effect in lowering plasma TG, but reduced significantly the plasma level of insulin and total cholesterol. Accordingly, LCMUFA exhibits a negative correlation with plasma levels of total cholesterol and insulin. However, LCMUFA is neutral when it comes to lowering plasma triglycerides.

Accordingly, LCMUFA and EPA seem to have complimentary effects on plasma lipids and insulin. The positive effect of LCMUFA (cetoleic acid) does therefore support the hypothesis that LCMUFA could have beneficial effect in reducing the risk of cardiovascular diseases. Intake of LCMUFA could as well have a positive impact on treating diabetes-2 by reducing non-HDL cholesterol and plasma insulin. Cardiovascular health from omega-3 is considered by many to be due to the effect of EPA. This is shown by success with the drug icosa-pent ethyl (Vascepa) containing 99% EPA (REDUCE-IT trial) and Epadel containing over 98% EPA (Jellis trial). One aim of the current invention is to provide a high concentration of cetoleic acid with low levels of EPA and DHA to allow easy informed choices for the customer. The product is however biased to EPA rather than DHA since EPA is considered more important for cardiovascular health.

Due to the complimentary effects of LCMUFA and EPA, the applicant is also interested in offering products for clinical purposes comprising of enriched cetoleic acid and EPA. The low amount of DHA will offer the option of making such products by mixing a concentrate of cetoleic acid and a concentrate of EPA. Production of such compositions is, however, more challenging if the LCMUFA fraction is already enriched in DHA. The disclosed method separates efficiently cetoleic acid from DHA which improves substantially the concentration of cetoleic acid, optionally retaining low levels of DHA.

In some embodiments, the combined concentration of EPA and DHA is in the range of 3.0-12.0 wt %, such as 5.0-12.0 wt %, more preferably 6.0-12.0 wt. % of the composition. In embodiments wherein the composition is enriched with EPA, the combined EPA and DHA concentration may be considerably higher.

The MUFA composition of the invention may, in addition to the cetoleic acid and EPA, and preferably gondoic acid, further comprise a variety of other fatty acids, including short and medium chain (C18 or less), long chain (C20-22), or even very long chain fatty acids (above C22), typically being present in the raw oil which the MUFA composition is derived from. These fatty acids may be saturated, monounsaturated or polyunsaturated. In some embodiments, the MUFA composition comprises short chain fatty acids. Naturally, however, the more concentrated the MUFA composition is in cetoleic acid, the lower the concentration of other fatty acids, including the shorter fatty acids, will be. Preferably, the content of short chain saturated fatty acids is kept low, particularly in the highly enriched cetoleic compositions of the invention. In some embodiments, the concentration of C12:1-C16:1 fatty acids is no more than maximum 2.0%. In some embodiments, the MUFA compositions comprises C18 fatty acids, such as:
C18:0, e.g. in the range of 0.5-2.7 wt. %;
C18:1, e.g. in the range of 5.0-16.0 wt. %
C18:2, e.g. in the range of 0.5-2.5 wt. %
C18:3, e.g. in the range of 0.5-2 wt. %.
C18:4, e.g. in the range of 0.5-1.5 wt. %

In one exemplary embodiment, an enriched LCMUFA composition of the invention comprises
C22:1 n11 (cetoleic acid) and C20:5 n3 (EPA), wherein;
the concentration of C22:1 n11 is at least 39 wt. %,
the concentration of EPA is at least 5.0 wt. %.

Furthermore, in specific embodiments, an enriched MUFA composition of the invention has one or more of the following characteristics:
the concentration of C22:1 n11 is at least 39 wt. %;
the C22:1 n11:C20:1 n9 weight ratio is from 1.5:1 to 2.6:1.
the concentration of EPA is at least 5.0 wt. %;
the concentration of DHA is no more than maximum 4.0 wt. %;
the concentration of C20:1 n11 (gadoleic) is no more than maximum 2.0 wt. %;
the concentration of C12:1-C16:1 fatty acids is no more than maximum 2.0%. In a preferred embodiment, the enriched MUFA composition fulfils all the characteristics above.

In analyses of the applicant, gas chromatography has been used, and GC area % gives information on fatty acid distribution of an analyzed composition. The fatty acid compositions provided preferably consist of close to 100% fatty acids, and there is hence only a negligible difference between the denominations “GC area %” used in analyses and examples, and the “% by weight” (wt. %) used throughout this specification and claims.

In certain embodiments, enriched MUFA compositions of the invention comprise the following fatty acids, within ranges as listed in Table A.

TABLE A

| Fatty acids | Common name | Range, wt % |
|---|---|---|
| 14:0 | Myristic acid | 0-3.0 |
| 16:0 | Palmitic acid | 0-4.0 |
| 16:1 n-7 | Palmitoleic acid | 0-2.0 |
| 16:4 n-1 | Hexadecatetraenoic acid | 0-0.6 |
| 18:0 | Stearic acid | 0.5-2.5 |
| 18:1 n-7 | Vaccenic acid | 0-7.0 |
| 18:1 n-9 | Oleic acid | 0.5-7.0 |
| 18:2 n-6 | Linoleic acid | 0.4-1.5 |
| 18:3 n-3 | alpha Linolenic acid | 0.5-1.0 |
| 18:4 n-3 | Stearidonic acid | 0-2.0 |
| 20:1 n-11 | Gadoleic acid | 1.0-2.5 |
| 20:1 n-9 | Gondoic acid | 12.0-28.0 |
| 20:4 n-6 | Arachidonic aicd | 0-0.5 |
| 20:4 n-3 | Eicosatetraenoic acid | 0-1.5 |
| 20:5 n-3 | Eicosapentaenoic acid (EPA) | 3.0-11.0 |
| 22:1 n-11 | Cetoleic acid | 39.0-60.0 |
| 22:1 n-9 | Erucic acid | 2.0-3.0 |
| 21:5 n-3 | Heneicosapentaenoic acid | 0-0.2 |
| 22:5 n-6 | Docosapentaneoic acid | 0-2.5 |
| 22:5 n-3 | Docosapentaneoic acid | 0.5-4.5 |
| 22:6 n-3 | Docosahexaenoic acid (DHA) | 0-4.0 |
| 24:1 n-9 | Nervonic acid | 1.0-3.0 |

In a further aspect, the invention provides a method for preparing a composition of C20-22 LCMUFAs, e.g. as according to the first aspect, wherein the method comprises steps to selectively separate LCPUFAs from LC-MUFAs enzymatically. The method comprises a step of selective transesterification by use of a lipase. In one embodiment, the method comprises the step of separating LCPUFAs from LCMUFAs by selectively cutting LCMUFAs from triglycerides enzymatically.

The method of the invention permits that MUFAs and PUFAs of the same length are separated from each other under mild conditions. More particularly, the MUFA C22:1 n11 (cetoleic acid) is separated from the PUFA C22:6 n3 (DHA). Accordingly, the method enables that fatty acids having similar boiling points, and hence are difficult to separate, are indeed selectively separated from each other. Other methods may enable the separation of C20 fatty acids from C22 fatty acids, but if concentrating e.g. C22:1 from C20 fatty acids, DHA (C22:6) will typically follow along with the C22:1 fatty acid.

Hence, in one embodiment the invention provides a method for preparing a composition of LCMUFAs, the method comprising the steps of:
i) mixing a natural oil, the natural oil comprising LCMUFAs and LCPUFAs, with a lipase to obtain a mixture comprising LCPUFAs on glyceride form and LCMUFAs on ethyl ester form;
ii) distilling the mixture of step i) to obtain an LCMUFA composition as one fraction and a LCPUFA composition as another fraction.

Furthermore, the invention provides a method for separating LCMUFAs from LCPUFAs, comprising the steps of:
i) mixing a natural oil with a lipase to obtain a mixture comprising LCPUFAs on glyceride form and LCMUFAs on ethyl ester form;
ii) distilling the mixture of step i) to obtain an LCMUFA composition as a first fraction and a LCPUFA composition as a second fraction.

In these methods, the enzymatic reaction takes place on the triglyceride, which is beneficial. As DHA fatty acids tend to be positioned on the middle position of the triglyceride, the fatty acids, e.g. LCMUFAs, positioned in the 1. and 3. can be selectively cleaved off by using a 1,3-specific lipase. DHA will hence be left on the glycerol, as a DHA monoglyceride (MAG-DHA). Hence, both regional selectivity and fatty acid selectivity is achieved.

In an alternative embodiment, also including transesterification by use of a lipase, the invention provides a method for separating LCMUFAs from LCPUFAs, comprising the steps of:
converting the fatty acids of a natural oil to ethyl esters;
distilling the ethyl esters to obtain fractions of LCMUFAs and LCPUFAs on ethyl ester form;
mixing the ethyl esters with glycerol and a lipase, obtaining a glyceride mixture comprising LCMUFAs. The DHA will remain as ethyl ester.

In the method of the invention, as disclosed above for the compositions of the invention, the start material is a natural oil comprising at least both cetoleic acid (LCMUFA) and EPA (LCPUFA). The starting oil, also called raw material, either being a crude oil or a refined oil used as starting oil for provision of the composition of the invention, typically comprises a least 9 wt. % cetoleic acid and at least 3 wt. % EPA, more preferably at least 15 wt. % cetoleic acid and at least 5 wt. % EPA, such as at least 7 wt. % EPA. The natural oil for use in the method, may hence be a raw oil, such as a raw oil typically produced in the fish meal production. Alternatively, the natural oil may be a refined oil, e.g. an oil where pollutants have been removed, such as which has gone through either of bleaching, deacidification, stripping or super critical extraction. The natural oil is from a marine source, such as from fish oil, squid oil, krill oil or algal oil, and preferably from fish oil. Particularly the oil is a North Atlantic fish oil, such as oil from herring, mackerel, capelin or tobis. Most preferably, the oil is oil from herring or mackerel, and most preferably from herring. The fatty acids of the start material is preferably on the form of triglycerides.

In step i) of the method an enzymatic separation is performed by using a lipase. Lipases are a family of enzymes that catalyze the hydrolysis of fats. Some lipases display broad substrate scope including esters of cholesterol, phospholipids, and of lipid-soluble vitamins. In the method of the invention, at least one lipase is used as an esterification catalyst to cut a fatty acid chain from the triglyceride backbone. As is known, lipases are well suited for use as catalysts in processes involving highly labile n-3 polyunsaturated fatty acids, such as EPA and DHA, occurring in marine oil. This is due to their ability to act at low temperatures, their neutral pH and their mildness in action, which helps keep to a minimum undesired side reactions such as cis-trans isomerizations, double-bond migrations, polymerizations, oxidations, etc. Thus, the utilization of lipases for the hydrolysis of fatty acids in marine oil is already well documented. The applicant has however unexpectedly found that certain lipases can be used selectively to separate MUFAs from PUFAs.

In step i) a lipase is brought into contact with the fatty acids of the natural oil, converting the LCMUFAs into LCMUFA esters. While the LCMUFAs are converted from triglyceride form to esters, the LCPUFAs will remain as glycerides. A mixture comprising LCPUFAs on glyceride form and LCMUFAs on ethyl ester form is hence obtained. The PUFAs of the mixture will typically comprise a mixture of mono-, di- and tri-glycerides. In one embodiment, ethanol is used as a substrate and the MUFAs of the mixture of step i) will be on the form of MUFA ethyl esters. No solvent is generally needed but can be used in some cases such as if the oil is unusually enriched in stearin.

The reaction conditions, including temperature, pressure and reaction time, are selected based on normal operation conditions used when converting triglycerides to ethyl esters by use of the same enzyme. The reaction with the lipase is typically to be run over some time, such as 1-48 hours, e.g. 20-30 hours, e.g. about 24 hours. Typically, a temperature in the range of 25-90° C. is appropriate, and a pressure of 1-50 mbar is appropriate if necessary.

A suitable amount of lipase will in most cases be about 10% (w/w) of the oil or less.

The method of the invention utilizes a lipase which is active to catalyze the esterification of MUFAs from the triglycerides. Suitable lipases are preferably immobilized enzymes, but also non-immobilised enzymes may work, although a more difficult after-use recovery is foreseen. In one embodiment, the lipase is a 1,3-specific lipase. Examples of useful lipases are the Rhizomucor miehei lipase (formerly named Mucor miehei lipase), Aspergillus niger lipase, Thermomyces lanuginosus lipase, Candida antarctica lipase, Candida rugosa lipase (formerly referred to as Candida cylindracea lipase), Geotrichum candidum lipase, Penicillium roguefortii lipase, Rhizopus delemar lipase, and Rhizopus oryzae lipase. More preferably the lipase is selected from the group of Rhizomucor miehei lipase, Thermomyces lanuginosus lipase, Candida antarctica lipase, and Candida rugosa lipase.

When the reaction of step i) is completed, the material may be cooled and filtered before step ii).

In step ii) the mixture of step i) is distilled to separate the LCPUFA glycerides of the mixture of step i) from the LCMUFA ethyl esters of this, obtaining an LCMUFA composition as a first fraction and a LCPUFA composition as a second fraction. In one embodiment, the distillation is a single column distillation, e.g. a single column short path distillation.

The obtained LCMUFA composition has a higher concentration of LCMUFAs, particularly of cetoleic acid, than in the natural starting oil. Furthermore, the obtained LCMUFA composition has only a very low amount of DHA present, as this C22 PUFA has been separated off from the C22 MUFAs and is found in the LCPUFA fraction (PUFA stream). Likewise, the LCPUFA composition has a higher concentration of LCPUFAs, than in the starting oil.

The method of the invention permits that MUFAs and PUFAs of the same length are separated from each other under mild conditions. More particularly, the MUFA C22:1 n11 (cetoleic acid) is separated from the PUFA C22:6 n3 (DHA). The obtained cetoleic rich first fraction from the method may be further concentrated to prepare an even more enriched MUFA composition or may be used as it is. The separated EPA-DHA-rich fraction (PUFA stream) may be also recovered, e.g. for commercial use. Hence, in a further aspect the invention provides a method for production of a MUFA composition according to the first aspect.

To illustrate what can be achieved by the method, as an example, starting from mackerel oil which comprises about 16 wt % cetoleic acid, 8% EPA and 12% DHA, an LCMUFA composition comprising about 21 wt % cetoleic acid can be obtained by the method of the invention, without any further enrichment performed. In such obtained LCMUFA stream, from enzymatic separation and one distillation (steps i and ii), the obtained EPA concentration is typically about 6%, while the DHA concentration is only 1.3%. Hence, from using the method of the invention, the cetoleic acid concentration of the LCMUFA composition is considerably increased at the same time as the DHA concentration has been considerably decreased. The applicant has found that the average change in concentration of cetoleic acid, from the raw oil, is about 2% when using the method of the invention. The invention accordingly provides a method for producing a composition of C20-22 LC-MUFAs. The obtained MUFA composition may be as disclosed in the first aspect, comprising C22:1 n11 (cetoleic acid) and C20:5 n3 (EPA) wherein;
the concentration of C22:1 n11 is at least 16 wt. %; and
the EPA concentration is at least 3 wt. %.

In one embodiment, the DHA concentration in the obtained composition is maximum 4.0 wt. % of the composition. In one embodiment, the EPA:DHA ratio is at least 4.0. Further embodiments of the obtained composition are as disclosed in the first aspect.

Equally, the method of the invention, separating LCMUFAs from LCPUFAs, further provides a method for producing an enriched composition of LCPUFAs. Starting from a North-Atlantic fish oil, such as e.g. mackerel oil, the PUFA-stream from the disclosed method (step i and ii), may typically comprise about 9-12 wt % EPA and 13-15% DHA. Accordingly, the method provides an efficient procedure for separating MUFAs from PUFAs and providing more concentrated compositions of such.

The obtained LCMUFA composition (“the LCMUFA stream”), from enzymatic separation and one distillation, is found valuable for example as an energy source, for use in food, feed or as source for provision of a more highly enriched LCMUFA composition. In one embodiment, the LCMUFA composition is for use in aquaculture, e.g. as part of fish feed. Further applications of the LCMUFA compositions of the invention are disclosed below, also including use in therapy or in health indications.

The obtained LCPUFA composition (“the LCPUFA stream”), from enzymatic separation and one distillation, is found valuable for example as an energy source, for use in feed or as source for provision of a more highly enriched LCPUFA composition. It may be a valuable alternative source of EPA and DHA to anchovy oil from a sustainable fish stock. These EPA and DHA can thus be further processed to diverse concentrates of EPA and DHA, and e.g. be used in nutraceuticals or pharmaceuticals.

The LCMUFA composition and LCPUFA composition obtained from step ii), may respectively be further concentrated to form more enriched compositions. Hence, in one embodiment, the LCMUFA first fraction composition (from step ii), is further concentrated to provide a more concentrated LCMUFA composition, i.e. a more enriched composition comprising a higher concentration of cetoleic acid. As an example, the separated mackerel oil disclosed above, comprising about 21 wt. % cetoleic acid, after steps i) and ii) may be further concentrated to increase the amount of cetoleic acid. As disclosed in the first aspect, compositions comprising at least 39.0 wt % cetoleic acid can accordingly be achieved. As shown in Examples 2 and 3, compositions comprising about 43-45 wt. % cetoleic acid have been obtained from mackerel or herring oil. Such further concentration to form enriched compositions of LCMUFAs may be accomplished employing processes such as distillation, extraction, enzymatic processing, chromatography and/or other fractionation methods known to one of skill in the art. Such further concentrating is preferably done by one or more distillations such as high quality molecular/short path distillation procedures. The method hence optionally further comprises a step iii) concentrating the wanted PUFAs or MUFAs, of the respective first and second fraction. Such concentrating may be accomplished employing processes such as distillation, extraction, enzymatic processing, chromatography, and is preferably accomplished by distillation, e.g. molecular distillation (short path distillation). In one embodiment, the method accordingly comprises an optional further step of concentrating the LCMUFAs, particularly the cetoleic acid, e.g. by distillation. Such optional further distillation(s) is hence a step towards making higher concentrates of cetoleic acid and still comprising low amount of DHA.

In one embodiment, step iii) comprises one or more rounds of distillation, each wherein the fraction comprising most cetoleic acid, is distilled again. By such process, compositions comprising at least 39 wt % cetoleic acid, such 39.0-60.0 wt % cetoleic acid, e.g. as disclosed in the first aspect, may be obtained. By the method of invention, e.g. including several distillations after the enzymatic separation, the cetoleic acid can be further purified and recovered in concentrations also above 50.0 wt. %, such as in concentrations of more than 60.0 wt. %, more than 70.0 wt. %, or even more than 80 wt. %.

In one embodiment, the invention provides a method for producing an enriched composition of C20-22 LC-MUFAs, the enriched composition comprising
C22:1 n11 (cetoleic acid) and C20:5 n3 (EPA), wherein;
the concentration of C22:1 n11 is at least 39 wt. %;
the concentration of EPA is at least 5.0 wt.

By using the enzymatic method of the invention, it surprisingly has been found possible to prepare a composition with a high concentration of cetoleic acid, the wanted range between cetoleic acid and gondoic acid, and the wanted EPA concentration. More particularly, the method enables the provision of a composition wherein the DHA concentration is low, and this is separated off from the MUFAs with the same length. Particularly, the provision of a composition comprising a high concentration of 20:1 and 22:1 MUFAs, and EPA concentration above 5%, combined with a very low DHA concentration, e.g. in the range of 0.5-3.5%, is achieved. Other methods, such as distillation alone, or even chromatography do not enable preparation of this combination (as exhibited by the examples below).

The method of preparation may further comprise steps to remove or reduce any toxic components. The purified and up-concentrated compositions of the invention further have a very low amount of unwanted pollutants. For instance, the amount of oligomeric and polymeric by-products, including oxidation products is reduced from the amount of such in the start oil. Furthermore, as the level of DHA is low in the composition, the tendency to oxidize is low, which is beneficial for the stability of the composition. Preferably, such oxidation products are at maximum 1.5%, such as maximum 1.0%, more preferably at maximum 0.5% by weight of the fatty acid composition. More particularly, the amount of environmental pollutants, like benzo(a)pyrene (BAP) and polyaromatic hydrocarbons (PAH), is low in the compositions of the invention. In one embodiment, the fatty acid mixture of the composition comprises less than 2 μg/kg of benzo(a)pyrene (BAP). In another embodiment, the composition preferably comprises less than 10 μg/kg of polyaromatic hydrocarbons (4PAH). 4PAH is defined as the sum of benz(a)anthracene, chrysene, benzo(b)fluoranthenes and benzo(a)pyrene. For measuring oxidation in oils there are two primary methods in use, to measure peroxide value and p-anisidine value.

The fatty acids of the compositions, both the MUFAs and PUFAs, and other fatty acids of the compositions, can be in different forms. In one embodiment, fatty acids of the composition are in a form selected from the group of free fatty acids; fatty acid salts; mono-, di-, triglycerides; esters, such as ethyl esters; wax esters; O-acetylated ω-hydroxy fatty acids (OAHFAs); cholesteryl esters; ceramides; phospholipids and sphingomyelins; alone or in combination. Or, the fatty acids may be in any form that can be absorbed in the digestive tract, or that can be absorbed by a bodily surface after topical application. Preferably, the fatty acids are in the form of free fatty acids, fatty acid salts, ethyl esters, or glycerides. In one embodiment, the LCMUFAs and LCPUFAs are independently selected from the group of free fatty acids, fatty acid esters and mono-, di- or triglycerides. In a preferred embodiment, the MUFAs of the MUFA composition of the invention are on the form of ethyl esters. If wanted, the form of the fatty acid can be converted to other forms. The skilled person would e.g. know how to transfer e.g. ethyl esters into free fatty acids or glycerides, and the method of producing may include such steps.

When referring to weight % of fatty acids in the mixture, any of the above broadest defined forms of the fatty acids may be used as basis for the calculation. Further, the fatty acids of the composition, provided in any of the forms listed above, are preferably not connected to other active ingredients. Accordingly, the fatty acid mixture of the composition is a pure, unreacted, highly concentrated MUFA and PUFA mixture. However, the fatty acid end groups may have been modified from the original, such as e.g. from glycerides to esters or free fatty acids, or opposite.

The LCMUFA compositions of the invention are useful in a range of different applications. The disclosed LCMUFA composition, in accordance with the first aspect, is found valuable for example as an energy source, for use in food, supplements, feed, in therapy, or as source for provision of a more highly enriched LCMUFA composition. Particularly, a MUFA composition of the invention from enzymatic separation and one distillation, the first fraction, comprising e.g. about 10-25% cetoleic acid, may be commercially interesting, e.g. for use in the food or feed industry.

In one aspect, the invention provides use of a MUFA composition as disclosed in the first aspect, comprising C22:1 n11 (cetoleic acid) and C20:5 n3 (EPA) wherein;
the concentration of C22:1 n11 is at least 16 wt. %;
the EPA concentration is at least 3 wt. %;
in food or feed. Further embodiments of the composition are as described for the first aspect.

In one exemplary embodiment, the MUFA composition of the invention is included in feed or food compositions that would normally comprise a certain amount of unsaturated fatty acids. As the claimed composition has a high stability, and not prone to oxidize, the claimed MUFA composition can beneficially replace oils used in food or feed which are more prone to oxidize. A further argument for using the MUFA composition in food, feed, or in supplements or nutraceuticals, is that cetoleic acid may act as a catalyzer for the conversion of ALA to EPA and DHA.

In another exemplary embodiment, the MUFA composition of the invention is included in feed, for instance for use in aquaculture, such as particularly in fish feed compositions, e.g. in feed pellets for fish farming. Particularly, the composition is for use in therapeutic feed for fish, e.g. for farmed salmon. In one example this is for preventive or therapeutic treatment of cardiac health.

In some embodiments, the invention relates to methods and compositions for treatment and alleviation of diseases or for use in health indications. As discussed in the background section, studies have been conducted, particularly by Yang et al, supporting that cetoleic acid, e.g. in combination with EPA, have beneficial health effects. The enriched MUFA composition of the invention, comprising a high concentration of cetoleic acid, as disclosed in the first aspect, may have one or more of the following health gains, providing: Decreased inflammation, improved TG and cholesterol, increased fat burning, decreased fatty liver, improve insulin sensitivity, improved metabolic syndrome. As indicated in Example 6, the composition of the invention may be used to affect metabolic syndromes positively. It may affect parameters relevant to diabetes (glucose tolerance, insulin sensitivity, glucose uptake in muscle), diabetic neuropathy—in particular allodynia (temperature sensitivity) and metabolic syndrome (liver fat content) and cardiovascular health (blood pressure).

In some embodiments, the composition is a nutraceutical, supplemental or pharmaceutical composition, and the composition is for use to treat, e.g. to prevent, reduce and/or alleviate the effects, symptoms, etc., of at least one health problem in a subject in need thereof. The MUFA composition is administered to the subject. In one embodiment, the composition is for use in maintaining good health, such as the maintenance of healthy cardiovascular system/heart health. In at least some embodiments of the present invention, the composition does not comprise an additional active agent. In this embodiment, the composition may be used in a pharmaceutical treatment of subject, such as of subjects diagnosed with a reduced level of MUFAs.

In one embodiment, the composition according to the invention is for use in human health, such as in the form of a supplement, i.e. a food, nutritional, or dietary supplement; a pharmaceutical, a medical food; or food for special medical purposes. In this latter group the composition may be selected from the group of Enteral Formulas for Special Medical Use, Foods for Specified Health Uses, Food for Special Medical Purposes (FSMP), Food for Special Dietary Use (FSDU), Medical Nutrition, and a Medical Food. Such a composition is particularly suitable for subjects having a deficiency of certain nutrients, such as of cetoleic acid. The composition is suited for a nutritional management of subjects having a distinctive nutritional requirement. Such a composition is typically administered to the subject under medical supervision. The composition comprises the relevant MUFAs, to increase or correct the level of the MUFAs in the blood or in specific tissue, such as of a subject diagnosed with a reduced ability for synthesis of MUFAs and/or with a low omega-3 index. The composition and the method of the invention have the ability to correct a nutritional deficiency in such a target population. In one exemplary use, the composition is for the maintenance of a healthy cardiovascular system/heart health. Particularly, the composition of the invention is for use as a dietary supplement for cardiovascular health.

The applicant is also exploring the potential beneficial role of the MUFA composition of the invention in skin health, which looks to be an important area in the future. In conditions where the skin lacks the ability to make MUFAs, the skin is characterised with poorly developed sebaceous glands which normally keep the skin from drying. This provides an indication of the importance of MUFA in skin biology. In one embodiment, the composition is for use in treating skin, such as in contributing to the skin's barrier function, keeping the skin appearing healthy, avoiding wrinkled skin or red spots, and also protecting against negative effects on the skin from the sun's UV radiation. Diseases and conditions related to the skin and hair that may be treated by the composition for use of the invention, comprise at least the following: dry and wrinkled skin, irritated, sour or sensitive skin, ability for wound healing, as protection (i.e. preventive treatment) against negative effects on the skin from the sun's UV radiation, negative effects on hair follicles, reduced hair health including risk of hair loss. Examples of skin diseases and conditions that typically give irritated/sour skin and which may benefit from treatment with the compositions for use are e.g. eczema, psoriasis, dermatitis, acne and rosacea (papulopustular rosacea). In one embodiment, the MUFA composition of the invention is included in a formulation for the skin, such as in a cosmetic product. By use of the composition or method of the invention one can normalize the fatty acid composition of a tissue, such as of the skin, such as by compensating for an abnormal sebaceous fatty acid composition. Such use or method may be seen as therapeutic or non-therapeutic.

Reference is made to Examples 4 and 5 below. Effect on skin of compositions of the invention, such as on human fibroblasts, may be seen as improved capacity of the cells to regenerate after damage (wound healing), to influence the production of melanin (skin pigment), or as reducing the expression of genes involved in inflammation (Ex. 4). In one embodiment, use of the composition results in an improvement of the grade and severity of eczema, such as atopic dermatitis (Ex. 5). Use of the composition may show changes in molecular markers such as lipid mediators, inflammatory markers and blood lipids. More specifically, use of the composition may positively affect one or more of the following: ceramide/lipid composition in skin, in inflammatory signalling molecules in skin, the omega-3 index in red blood cells, cetoleic acid in red blood cells, cholesterol/blood lipid profile, trans-epithelial water loss (TEWL), hydroxylated omega-3 fatty acids in plasma, the association of omega-3 index with Eczema area and severity index (EASI) parameters, the association of cetoleic acid content in red blood cells with EASI parameters.

Accordingly, in one embodiment, the invention provides the composition of the first aspect for use as a nutraceutical, food supplement, food additive or cosmetic product.

Compositions according to the invention, such as supplements, may be delivered or administered in any suitable format, including, but not limited to, oral delivery, dermal delivery or mucosal delivery, including as eye drops. The composition of the invention along with other ingredients can be formulated to include acceptable excipients and/or carriers for oral consumption, and in particular in the form of an oral delivery vehicle, such as capsules, preferably gelatine capsules, liquids, emulsions, tablets or powders.

Accordingly, the invention provides a MUFA composition as disclosed in the first aspect, comprising C22:1 n11 (cetoleic acid) and C20:5 n3 (EPA) wherein;
the concentration of C22:1 n11 is at least 16 wt. %;
the EPA concentration is at least 3 wt. %
for use in therapy or health indications.

Likewise, the invention provides a method of treatment, administering to a subject a MUFA composition as disclosed in the first aspect, comprising C22:1 n11 (cetoleic acid) and C20:5 n3 (EPA) wherein;
the concentration of C22:1 n11 is at least 16 wt. %;
the EPA concentration is at least 3 wt. %.

In some embodiment, the composition for use is an enriched LCMUFA composition comprising;
C22:1 n11 (cetoleic acid) and C20:5 n3 (EPA), wherein;
the concentration of C22:1 n11 is at least 39 wt. %;
the concentration of EPA is at least 5.0 wt.

Hence, the invention provides a composition according to the first aspect, for use as a supplement, a pharmaceutical, medical food or as food for special medical purposes, or for the skin.

The MUFA composition for use is as detailed in the first aspect. Particularly, the composition for use in therapy, or for use in a method of treatment, is for use in either of decreasing inflammation, improving TG and cholesterol, increasing fat burning, decreasing fatty liver, or improve insulin sensitivity or improving metabolic syndrome, which are indications wherein cetoleic acid and EPA have combined benefits. Furthermore, the composition of the invention may be for use in one or more of reducing triacylglycerides, increasing HDL cholesterol, decreasing blood pressure, decreasing inflammation, and as precursor for specialized proresolving mediators (SPMs). Furthermore, the composition of the invention is for use in one or more of decreasing non-HDL cholesterol, decreasing hyperinsulinaemia, glucose regulation, reduction inflammation, which are effects of cetoleic acid. With reference to the studies of Yang et all, 2016, the MUFA composition of the invention has been developed with specific therapeutic benefits in mind. Firstly, in reducing the risk of cardiovascular disease; and secondly as an alternative means of providing omega-3 levels in humans, thereby offering benefits in areas such as chronic inflammation, cognition and healthy ageing. The cell culture studies and human clinical studies of Østbye et al, BR, J Nutr. Oct. 14, 2019, 122(7), 755-768, support the hypothesis that fish-oil rich in cetoleic acid promotes the conversion of α-linolenic acid to EPA, supporting the use of cetoleic-rich oils as the claimed composition as a relevant source of EPA and DHA. In one embodiment, the composition is for use in treatment of one or more of metabolic syndrome, fatty liver disease (NAFLD, NASH), diabetes, prediabetes and cardiovascular disease.

In a further aspect, the invention provides a combined formulation. Such formulation may be a combination of a MUFA composition as disclosed in the first aspect, with another marine oil. As one alternative, the MUFA composition of the invention is combined with an anchovy oil or fractions from this, e.g. to further concentrate the composition with PUFAs, providing formulations useful for different type of applications.

The invention shall not be limited to the shown embodiments and examples. While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is to be understood that various alternatives to the embodiments described herein can be employed in practicing the disclosure.

It is to be understood that every embodiment of the disclosure can optionally be combined with any one or more of the other embodiments described herein.

It is to be understood that embodiments disclosed for one aspect also apply to other aspects of the invention. For instance, the embodiments disclosed for the compositions also apply for the aspect directed to the method for production and to the compositions for use.

It is to be understood that each component, compound, composition, or parameter disclosed herein is to be interpreted as being disclosed for use alone or in combination with one or more of each and every other component, compound, composition or parameter disclosed herein. It is further to be understood that each amount/value or range of amounts/values for each component, compound, or parameter disclosed herein is to be interpreted as also being disclosed in combination with each amount/value or range of amounts/values disclosed for any other component(s), compound(s), or parameter(s) disclosed herein, and that any combination of amounts/values or ranges of amounts/values for two or more component(s), compound(s), or parameter(s) disclosed herein are thus also disclosed in combination with each other for the purposes of this description. Any and all features described herein, and combinations of such features, are included within the scope of the present invention provided that the features are not mutually inconsistent.

It is to be understood that each lower limit of each range disclosed herein is to be interpreted as disclosed in combination with each upper limit of each range disclosed herein for the same component, compound, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, or parameter disclosed in the description or an example is to be interpreted as a disclosure of either a lower or an upper limit of a range and thus can be combined with any other lower or upper limit or a range or specific amount/value for the same component, compound, or parameter disclosed elsewhere in the application to form a range for that component, compound, or parameter.

### EXAMPLES

### Example 1. Comparison—Double Stage Distillation Versus Use of Lipase Process According to the Invention

The starting oil in this example was mackerel oil which had been deacidified (to remove free fatty acids) and stripped by single column short path distillation to remove organic pollutants. The refining is optional and could be made in different ways like up-front bleaching, employing supercritical extraction, membrane filtration etc. The same comment applies to the other examples.

Batch 1, for comparison, was made by ethylating the fish oil (starting oil) by chemical ethylation of the fish oil, using 2% sodium ethoxide/ethanol in quantitative yield of ethyl ester (>95%). The work-up was traditional by removing glycerol after evaporating off excess ethanol followed by washing the ethyl esters with water and citric acid. The esters were distilled by using VTA short path distillation pilot plant (VK 83-6) comprised of two columns of 0.06 m² area and 0.285 m in height. The flow rate was adjusted to about 4 kg/hr and the temperature 115° C. on the first column whereas 165° C. on the second column. The pressure was less than 10⁻³ mbar during the distillation. The distillation was tuned to remove mostly short chain fatty acids (C14-C16) to preserve the content of the long chain fatty acids (table 1).

Batch 2 was made by selective transesterification according to the invention using a 1,3 regiospecific lipase using stochiometric amount of ethanol. The lipase was 2% (w/w) of the oil. When a target conversion was reached (varies), the lipase was filtered off. Excess ethanol was removed, and the ethyl esters collected by a single column stripping using the same VTA pilot plant as described above. The temperature on the stripping column was 165° C. and pressure less than 10⁻³ mbar.

The results from both batches are exhibited in Table 1 below. It is noted that the composition of gondoic acid and cetoleic acid was quite similar for both batches, 12-13% (121-123 mg/g) and 20-21% (199-203 mg/g), respectively. However, substantial difference was seen in the amount of EPA and DHA between the batches. In batch 1 the sum EPA+DHA was 26% and EPA/DHA was 0.68 which is a similar ratio as found in the starting oil. In batch 2, using lipase, the sum EPA+DHA was only 7% and the ratio EPA/DHA was 4.4 which is more than a six-fold increase from the starting oil. Cetoleic acid/DHA was 16.04 which is more than a 11-fold increase from the starting oil.

The results provided in the table further demonstrate that the amount of short chain fatty acids (C14-C16) is about 27% (w/w) when employing the lipase compared to 7% in the case of using only distillation. This is a significant advantage of an intermediate which enables further concentration by distillation as these short chain fatty acids have considerably lower boiling point which will be reflected in the overall yield of oil. The example demonstrates that by employing 1,3-regiospecific lipase, characterized by certain fatty acid selectivity, one can achieve similar composition of LCMUFAs as compared to distillation, however, comprising much less EPA+DHA and even less than expected DHA as compared to EPA. A person skilled in the art will recognize that separation of fatty acids, or derivatives thereof, can be achieved either due to their polarity (unsaturation) or boiling point. A skilled person will realize that separating LCMUFA such as cetoleic acid and gondoic acid from EPA and DHA is non-trivial and the results achieved by this example are surprising and novel.

TABLE 1
comparison of lightly distilling ethyl ester of mackerel
oil by short path distillation after non-regiospecific chemical
ethylation (2% sodium ethoxide/ethanol) versus ethyl esters
made by selective transesterification with ethanol using
1,3-regiospecific lipase (method of invention).

| | | Batch 1: | |
|---|---|---|---|
| | | (GC area %) | Batch 2: |
| | Mackerel oil | Distillation | (GC area %) |
| Fatty acid | (area % GC | comparison | Lipase process |
| 14:0 | 7.1% | 0.0% | 8.2% |
| 16:0 | 12.9% | 5.5% | 14.7% |
| 16:1 n-7 | 4.3% | 1.1% | 4.6% |
| 16:4 n-1 | 0.5% | 0.0% | 0.0% |
| 18:0 | 2.1% | 2.4% | 2.7% |
| 18:1 n-7 | 10.6% | 11.1% | 12.5% |
| 18:1 n-9 | 2.0% | 2.2% | 2.1% |
| 18:2 n-6 | 1.7% | 1.8% | 2.0% |
| 18:3 n-3 | 1.4% | 1.5% | 1.7% |
| 18:4 n-3 | 4.6% | 4.6% | 2.9% |
| 20:1 n-11 | 0.6% | 0.8% | 0.8% |
| 20:1 n-9 | 9.8% | 12.6% | 12.3% |
| 20:4 n-6 | 0.0% | 0.5% | 0.0% |
| 20:4 n-3 | 1.1% | 1.4% | 0.8% |
| 20:5 n-3 | 8.3% | 10.5% | 6.5% |
| 22:1 n-11 | 16.1% | 21.0% | 20.5% |
| 22:1 n-9 | 1.0% | 0.7% | 1.3% |
| 21:5 n-3 | 0.5% | 0.6% | 0.0% |
| 22:5 n-6 | 0.3% | 0.6% | 0.0% |
| 22:5 n-3 | 1.4% | 1.9% | 0.0% |
| 22:6 n-3 | 11.9% | 15.4% | 1.3% |
| 24:1 n-9 | 1.0% | 1.5% | 1.4% |
| Yield of oil (EE) | — | 69% | 64% |
| 20:1 + 22:1 | 25.9% | 33.5% | 32.9% |
| EPA + DHA | 20.2% | 25.9% | 7.8% |
| 20:1 n-9, mg/g | 96 | 123 | 121 |
| 22:1 n-11, mg/g | 156 | 203 | 199 |
| gondoic/cetoleic | 0.61 | 0.60 | 0.60 |
| EPA/DHA | 0.70 | 0.68 | 5.11 |
| C14-C16 | 24.8% | 6.6% | 27.6% |
| C18 | 22.4% | 23.5% | 23.8% |

### Example 2. Concentration of Cetoleic Acid by Distillation with or without the Use of Lipase

This example demonstrates how ethyl esters made by selective ethylation, according to the invention, was surprisingly more effective in concentrating cetoleic acid than compared to non-selective ethylation. The two batches made in example 1 were distilled further using the same pilot plant as in example 1. Fractions were collected and analyzed by GC for estimating mg/g and % area of key fatty acids. The results are exhibited in the Table 2 below.

When concentrating batch 2 and batch 1 from example 1, fractions A (selective/lipase) and C (non-selective/chemical) were made in comparable yield of oil or 45% vs 51% respectively. The sum of 20:1+22:1 was similar in both cases, 47% and 42% respectively. It is noted a striking difference between the methods both in terms of EPA+DHA, as well as the ratio of cetoleic acid/DHA; 11% and 12.23 in the case of fraction A compared to 31% and 1.37 in the case of fraction C. Secondly, the fraction A is still substantially richer in short chain fatty (C14-C16 and C18) acids than C which favours further enrichment of cetoleic acid. Batch 2 (lipase) was thus concentrated further resulting in fraction B obtained in an overall yield of 31%. This fraction had similar amount of short chain fatty acids as seen in C (9.8% vs 13.9%). Fraction B, however, is much richer in cetoleic acid than found in fraction C, 44% cetoleic acid EE (or 423 mg/g as EE) compared to only 27% (265 mg/g as EE) for the non-selective process. It is still retained only 12% EPA+DHA in fraction B and the high ratio of cetoleic acid/DHA, or 13.08 which is 9.5-fold higher than found in fraction C and 9.6-fold higher than found in the starting oil.

It is further noted from Table 2 that the yield of cetoleic acid was comparable for fraction B and C, 83% and 87% respectively, even though B contains about 1.6-fold higher amount of said fatty acid. This demonstrates that the lipase method of the invention is more efficient in making high concentrates of cetoleic acid but still keeping low amount of EPA+DHA and high ratio of cetoleic acid/DHA. The ratio of gondoic acid/cetoleic acid retains similar as seen in the starting oil.

A person skilled in the art will recognize that comparison fraction C could be distilled further to similar content of cetoleic acid as found in fraction B. But that would lead to substantially less yield of oil and cetoleic acid. Further distillation of C will lead to proportional increase in the content of DHA and cetoleic acid due to similar boiling point of these fatty acid esters. Such concentrate of cetoleic acid would thus be rich in cetoleic acid and DHA, and have a low ratio of cetoleic acid/DHA. This example hence demonstrates that using a 1,3 regio-specific lipase, characterized by certain fatty acid selectivity, is surprisingly more effective in concentrating cetoleic acid than distillation.

TABLE 2
comparison of making concentrate of ethyl ester of cetoleic acid by short path distillation
after non-regiospecific chemical ethylation (2% sodium ethoxide/ethanol) vs ethyl esters
generated by selective transesterification with ethanol using 1,3-regiospecific lipase.

| | | | | | Only distillation | |
|---|---|---|---|---|---|---|
| | | 1,3 regio-specific lipase + | | | (comparison): | |
| | Mackerel oil | distillation: different | | | different split | |
| | (GC area %) | split (GC area %) | | | (% a-GC) | |
| | Starting | Batch 2: | | | Batch 1: | |
| Fatty acid | oil | Ex 1 | A | B | Ex 1 | C |
| 14:0 | 7.1% | 8.2% | 0.1% | 0.0% | 0.0% | 0.0% |
| 16:0 | 12.9% | 14.7% | 4.1% | 0.2% | 5.5% | 0.3% |
| 16:1 n-7 | 4.3% | 4.6% | 0.0% | 0.0% | 1.1% | 0.0% |
| 16:4 n-1 | 0.5% | 0.0% | 3.2% | 0.0% | 0.0% | 0.0% |
| 18:0 | 2.1% | 2.7% | 0.6% | 1.7% | 2.4% | 1.8% |
| 18:1 n-7 | 10.6% | 12.5% | 0.6% | 5.7% | 11.1% | 6.6% |
| 18:1 n-9 | 2.0% | 2.1% | 14.5% | 0.2% | 2.2% | 1.3% |
| 18:2 n-6 | 1.7% | 2.0% | 1.9% | 0.7% | 1.8% | 1.0% |
| 18:3 n-3 | 1.4% | 1.7% | 1.8% | 0.7% | 1.5% | 0.9% |
| 18:4 n-3 | 4.6% | 2.9% | 1.5% | 0.5% | 4.6% | 2.1% |
| 20:1 n-11 | 0.6% | 0.8% | 1.1% | 1.3% | 0.8% | 1.0% |
| 20:1 n-9 | 9.8% | 12.3% | 16.9% | 19.5% | 12.6% | 14.9% |
| 20:4 n-6 | 0.0% | 0.0% | 0.0% | 0.0% | 0.5% | 0.5% |
| 20:4 n-3 | 1.1% | 0.8% | 1.1% | 1.2% | 1.4% | 1.6% |
| 20:5 n-3 | 8.3% | 6.5% | 8.8% | 8.4% | 10.5% | 11.2% |
| 22:1 n-11 | 16.1% | 20.5% | 30.1% | 43.6% | 21.0% | 27.3% |
| 22:1 n-9 | 1.0% | 1.3% | 1.9% | 2.2% | 0.7% | 1.4% |
| 21:5 n-3 | 0.5% | 0.0% | 0.0% | 0.0% | 0.6% | 0.9% |
| 22:5 n-6 | 0.3% | 0.0% | 0.0% | 0.0% | 0.6% | 0.5% |
| 22:5 n-3 | 1.4% | 0.0% | 1.4% | 2.0% | 1.9% | 2.6% |
| 22:6 n-3 | 11.9% | 1.3% | 2.5% | 3.3% | 15.4% | 19.9% |
| 24:1 n-9 | 1.0% | 1.4% | 2.0% | 2.7% | 1.5% | 2.1% |
| Yield of oil (EE) | — | 64% | 45% | 31% | 69% | 51% |
| 20:1 + 22:1 | 25.9% | 32.9% | 47.0% | 63.1% | 33.5% | 42.2% |
| EPA + DHA | 20.2% | 7.8% | 11.2% | 11.7% | 25.9% | 31.0% |
| 20:1 n-9, mg/g | 96 | 121 | 166 | 191 | 123 | 146 |
| 22:1 n-11, mg/g | 156 | 200 | 292 | 423 | 203 | 265 |
| Yield (cetoleic | | 83% | 83% | 83% | 90% | 87% |
| acid | | | | | | |
| gondoic/cetoleic | 0.61 | 0.60 | 0.56 | 0.45 | 0.60 | 0.54 |
| EPA/DHA | 0.70 | 5.11 | 3.57 | 2.51 | 0.68 | 0.56 |
| C14-C16 | 24.8% | 27.6% | 7.4% | 0.2% | 6.6% | 0.3% |
| C18 | 22.4% | 23.8% | 20.9% | 9.6% | 23.5% | 13.6% |

### Example 3. Concentration of Cetoleic Acid by Using Herring Oil and 1,3 Regiospecific Lipase

Refined herring oil was treated with selective transesterification using 1,3 regiospecific lipase and using stochiometric amount of ethanol and lipase. The lipase was filtered off when the target conversion was reached (varies). Excess ethanol was removed and the ethyl esters collected by a single column stripping at 165° C., Fraction A. The distillate was further distilled by using the same VTA two column shorth path distillation plant as used in example 1. The pressure was less than 10-3 mbar under the distillation process and the temperature of the columns was between 10° and 140° C. during double stage distillation (concentration of cetoleic acid). The results can be seen in Table 3 below.

After distillation of fraction A, an oil comprising 44% cetoleic acid (434 mg/g EE) was harvested comprising only 10% of EPA+DHA and a ratio of 4.72 of EPA/DHA which is 5.2-fold higher than found in the refined herring oil. The ratio of cetoleic acid/DHA was increased as well significantly from 2.22 in the starting oil to 26.49 in the 44% concentrate of cetoleic acid, which is about 12-fold increase. The ratio of gondoic acid and cetoleic acid is similar as seen in the starting oil. The process therefore preserves said ratio while concentrating cetoleic acid. Hence, the method of the invention can be used for diverse types of fish oil which are rich in cetoleic acid.

TABLE 3
Selective transesterification of refined herring oil using
1,3-regio-specific lipase and following distillations.

| | | Stripped oil | Distillation |
|---|---|---|---|
| | Herring oil | (GC area %) | of (A) (GC |
| Fatty acid | (GC area %) | Fraction A | area %) |
| 14:0 | 7.5% | 7.8% | 1.2% |
| 16:0 | 13.2% | 13.4% | 1.2% |
| 16:1 n-7 | 5.2% | 6.1% | 1.1% |
| 16:4 n-1 | 0.5% | 0.2% | 0.5% |
| 18:0 | 1.2% | 1.4% | 0.9% |
| 18:1 n-7 | 11.7% | 13.6% | 3.9% |
| 18:1 n-9 | 1.4% | 1.9% | 0.8% |
| 18:2 n-6 | 1.5% | 1.6% | 1.1% |
| 18:3 n-3 | 1.1% | 1.0% | 1.0% |
| 18:4 n-3 | 3.2% | 1.3% | 2.3% |
| 20:1 n-11 | 1.1% | 1.3% | 1.7% |
| 20:1 n-9 | 12.5% | 15.6% | 23.6% |
| 20:4 n-6 | 0.0% | 0.1% | 0.3% |
| 20:4 n-3 | 0.5% | 0.2% | 0.6% |
| 20:5 n-3 | 7.7% | 3.8% | 8.0% |
| 22:1 n-11 | 18.9% | 23.4% | 44.8% |
| 22:1 n-9 | 0.9% | 1.2% | 2.3% |
| 21:5 n-3 | 0.3% | 0.0% | 0.6% |
| 22:5 n-6 | 0.0% | 0.0% | 0.0% |
| 22:5 n-3 | 0.7% | 0.2% | 0.6% |
| 22:6 n-3 | 8.5% | 1.0% | 1.7% |
| 24:1 n-9 | 0.9% | 1.1% | 1.1% |
| Yield of oil (EE) | — | 62% | 29% |
| 20:1 + 22:1 | 31.3% | 39.0% | 68.4% |
| EPA + DHA | 16.2% | 4.8% | 9.7% |
| 20:1 n-9, mg/g | 122 | 153 | 231 |
| 22:1 n-11, mg/g | 183 | 227 | 434 |
| Yield (cetoleic | | 77% | 69% |
| acid) | | | |
| gondoic/cetoleic | 0.66 | 0.66 | 0.53 |
| EPA/DHA | 0.90 | 3.65 | 4.72 |
| C14-C16 | 26.5% | 27.6% | 4.0% |
| C18 | 20.0% | 20.8% | 10.0% |

### Example 4. Study Using Human Fibroblasts to Determine Effect of LCMUFA Concentrate on Skin Health Including Wound Healing

Different fish oils contain varying amounts of omega-3 fatty acids and other less well-known fatty acids. North Atlantic fish oils contain lower levels of EPA and DHA than South-American fish oils, but are richer in the long-chained mono-unsaturated fatty acids (LCMUFA) particularly C22:n1-11 (Cetoleic acid) which is unique to marine oils. Compared to EPA and DHA there is a paucity of research into the uptake, incorporation into tissue and health benefits of supplementation with marine LCMUFAs. Earlier studies (Huang et al, Cosmetic and Therapeutic Applications of Fish Oil's Fatty Acids on the Skin. Mar. Drugs 2018, 16, 256, have shown that fish-oil derived fatty acids taken as an oral supplement can improve skin barrier function, reduce UV-induced inflammation, reduce unwanted hyperpigmentation and improve wound healing. Several studies indicate a positive effect of fish oil and omega-3 on skin diseases, however, there are also several contradictory reports from meta-analyses. The contradictory results can possibly be explained by the complex components and composition of fish oils, in addition the quality of the oils can vary according to the fish species used and the manufacturing process. Both the LC-MUFA and omega-3 content of fish oils are important factors when determining health effects of fish oils. Studies performed by the applicant and NOFIMA in rats and salmon show that for both these species the highest concentration of the fatty acid C22:1n-11 (Cetoleic acid) is seen in skin.

Preliminary cell studies with human fibroblasts have been performed to test potential health benefits of LCMUFA in skin.

The main objective of the study was to show whether addition of a LCMUFA concentrate (Cetoleic acid concentrate) or DHA to cell culture medium would:
1) Influence the capacity of the cells to regenerate after damage (so called “wound healing model”),
2) Influence the production of melanin (skin pigment),
3) Influence the expression of genes involved in inflammation after induced oxidative stress.

The LCMUFA concentrate used in the study was a North-Atlantic fish oil concentrate, prepared from mackerel oil, comprising:
Cetoleic acid, C22:1: 48.74%,
DHA, C22:6:n3: 3.96%,
C20:1:18.84%
Given as GC area %.

In the test material used in this study the concentration of EPA, C20:5 n3, was 0.80 GC area %.

Wound healing model: Treatment arms included addition of DHA or the LCMUFA contrate to the cell culture medium, respectively. The control was cells only added culture medium, no fatty acids. The effect on closing of wound 14 hours after initiation of a wound was measured.

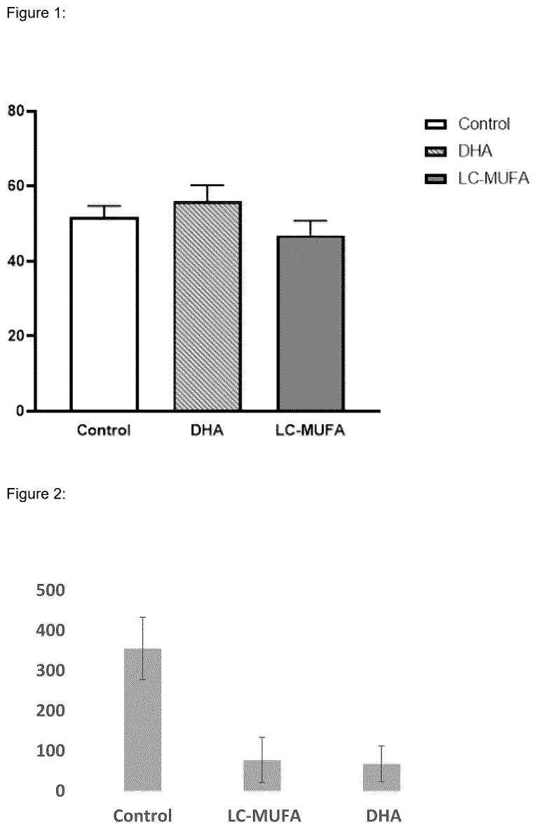

Results are shown in the Figures. For FIG. 1, the Y-axis shows the scratch wound area %, and the x-axis provides bars for control, DHA and the LCMUFA concentrate. The initial wound area was defined as 100%, then the wound opening was measured after 14 hours to a percentage of the original wound. The reduction was approximately 60% for LC-MUFA and 50% for DHA, respectively. Accordingly, a trend was seen for improved wound healing in the LCMUFA study arm.

Menadion was added to cell culture medium to induce oxidative stress. Melanin production was reduced in both the LCMUFA and DHA treated cells, please see FIG. 2 showing melanin concentration after oxidative stress.

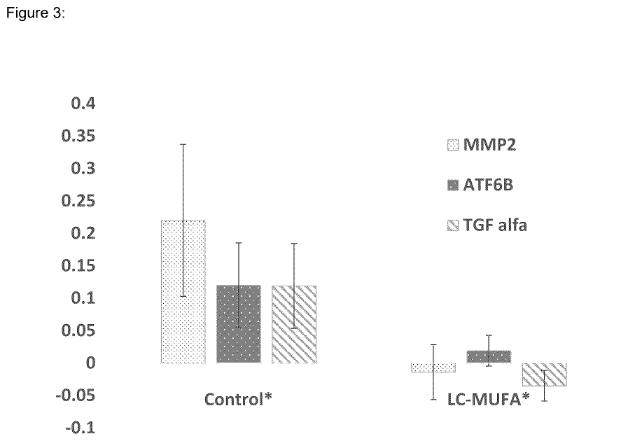

Expression of genes related to inflammation showed no statically significant difference between treatment groups, but a trend was seen for lower expression of the genes MMP2, ATF6B and TNF-alpha in the LCMUFA arm compared to control, please see FIG. 3. The DHA treated cells showed no such tendency, and data is not shown.

Conclusion: Preliminary data from cell studies indicates a positive effect of LCMUFA concentrate on skin health, in particular to unwanted hyperpigmentation due to reduced melanin synthesis.

### Example 5. a Randomized, Double-Blinded Nutritional Study to Determine the Effect of a Concentrated Cetoleic Acid Fish Oil on Atopic Dermatitis

A study is being conducted to primary determine if a daily intake of 2 g of the investigational product “EPAX Cetoleic 30” changes the grade and severity of those with mild to moderate atopic dermatitis as assessed by Eczema Area and Severity Index (EASI). Secondary, the objective is to determine if a daily intake of 2 g “EPAX Cetoleic 30” is associated with changes in molecular markers (lipid mediators, inflammatory markers and blood lipids) in those with mild to moderate atopic dermatitis as compared to placebo.

### Investigational Product:

A North-Atlantic fish oil concentrate, (EPAX Cetoleic 30), containing at least 300 mg/g cetoleic acid. Specifically, the product comprises the following fatty acids, given in weight % and as GC area %:
EPA (C20:5): 5.7 wt %, 6.12 area %
DHA (C22:6): 1.6 wt %, 1.66 area %
gondoic acid (C20:1 n9): 21.6 wt %, 23.16 area %
cetoleic acid (C22:1 n11): 41.5 wt %, 45.46 area %

The total LCMUFA weight % is 67.6% (TG).

The investigational product was prepared by the claimed method.

The product is provided in the form of 1 gram capsules. Subjects will take 2×1 g capsules daily for 6 months. The placebo is corn oil.

The study population is based on volunteers (age 18-80) with mild to moderate atopic dermatitis, Eczema area and severity index (EASI) between 1.1-21.0. The project will use medical records from a Norwegian hospital for pre-screening of subjects with mild to moderate atopic dermatitis. The study population will be block randomised to ensure equal representation of medicated vs non-medicated volunteers in each arm.

The study is double blinded. Subjects will be assigned to one of the 2 nutritional groups according to a randomization scheme, with 30 subjects in each group.

Group 1: Placebo: Corn-oil capsules.

Group 2: Study supplement: EPAX Cetoleic 30-capsules

Inclusion criteria: subjects who are diagnosed with active atopic dermatitis, with an EASI of 1.1-21.0, who are willing to refrain from oil supplements (omega-3, borage, evening primrose, etc.) for 1 month before study begin (washout) and during the study, and who are willing to take the study supplement for 6 months.

Study visits will include clinical evaluation, blood sampling, skin sampling, and subject evaluation (questionnaire) on day 1, after 6 weeks, 3 months and 6 months.

### Samples and Analysis:

Clinical evaluation: Eczema area and severity index (EASI)
Patient reported evaluation: Itch Numerical Rating Scale (NRS), Dermatology Life Quality Index (DLQI), Patient Oriented Eczema Measure (POEM), use of topical treatment.
Skin tape sample (10 consecutive tapes): ceramides, cytokines
Skin barrier: trans-epithelial water loss (TEWL)
Blood sample (Red blood cells (RBCs) and plasma): omega-3 index, hydroxylated omega-3s
Blood sample for blood lipids (cholesterol-package) and liver enzymes (ALAT, ASAT, GTT)

The primary efficacy variable will be the change in clinical and subject evaluation parameters for assessment of eczema.

The secondary efficacy variables are:
The change of ceramide/lipid composition in skin
The change in inflammatory signalling molecules in skin
The change of omega-3 index in red blood cells
The change in cetoleic acid in red blood cells
The change in cholesterol/blood lipid profile
The change in trans-epithelial water loss (TEWL)
The change of hydroxylated omega-3 fatty acids in plasma
The association of omega-3 index with EASI parameters
The association of cetoleic acid content in RBCs with EASI parameters

When the study has been finalized, it is expected that for Group 2, the subjects receiving the cetoleic rich capsules, at least one of the parameters for assessment of eczema will show a favourable change, compared to the Group 1 receiving the placebo.

### Example 6. Animal Study to Assess Effects on Metabolic Syndrome

A study was performed with ZDSD rats, an obese-diabetic rat model that spontaneously develop type 2 diabetes. The rats were fed for 6 weeks with a feed supplemented with herring oil (comprising cetoleic acid) to assess how it affects metabolic syndrome.

It was found that the supplementation of feed with the herring oil improved insulin sensitivity and glucose levels compared to a soya control. These parameters are key targets for diabetes regulation and metabolic syndrome.

Future studies are also planned with Zucker fa/fa rats which are naturally pre-diabetic, meaning they have mild insulin insensitivity and increased fasting glucose levels but less than that seen with full diabetes. Rats will be divided into a low cetoleic acid group (herring oil), high cetoleic acid group (EPAX Cetoleic 30, the same investigational product as used in Example 5) and a soya control group and dosed for 6 weeks. The study will measure parameters relevant to diabetes (glucose tolerance, insulin sensitivity, glucose uptake in muscle,) diabetic neuropathy—in particular allodynia (temperature sensitivity) and metabolic syndrome (liver fat content) and cardiovascular health (blood pressure).

1. A composition enriched in long chain monounsaturated fatty acids (LCMUFAs), the composition comprising C22:1 n11 (cetoleic acid) and C20:5 n3 (EPA),
wherein the fatty acids of the composition are derived from a natural oil comprising cetoleic acid and EPA, and wherein
the concentration of cetoleic acid is at least 16.0 wt. %;
the EPA concentration is at least 3 wt. %;
any concentration of DHA is maximum 4 wt. %, and
wherein the EPA:DHA ratio is at least 4:1, and
the composition comprising the fatty acids in form of ethyl esters, free fatty acids or fatty acid salts.

2. The LCMUFA composition of claim 1 derived from fish oil, particularly from oil of herring, mackerel, capelin, cod, saithe or tobis.

3. The LCMUFA composition of claim 1 comprising 16.0-60.0 wt % cetoleic acid.

4. The LCMUFA composition of claim 1 further comprising at least one C20:1 fatty acid, and wherein the weight ratio between C22:1 and C20:1 fatty acids is from 1.5:1 to 2.6:1.

5. The LCMUFA composition of claim 1 comprising EPA in a concentration of 5.0-40.0 wt. % of the composition.

6. The LCMUFA composition of claim 1 comprising gondoic acid, C20:1 n9, in a range of 9-28 wt % of the composition.

7. The LCMUFA composition of claim 1, wherein the cetoleic acid:DHA ratio is at least 10:1.

8. The LCMUFA composition of claim 1 wherein:
the concentration of cetoleic acid is at least 39%, and
the concentration of EPA is at least 5.0%.

9. The LCMUFA composition of claim 1 wherein the combined concentration of EPA and DHA is in the range of 6.0-12.0 wt. % of the composition.

10. The LCMUFA composition of claim 1 wherein the cetoleic acid:DHA ratio is at least 15:1.

11. A method for preparing a composition enriched in long chain monounsaturated fatty acids (LCMUFA) comprising at least 16 wt. % cetoleic acid (C22:1 n11), from a natural oil comprising LCMUFAs including cetoleic acid, and long chain polyunsaturated fatty acids (LCPUFAs),
the method comprising the steps of:
i) selective transesterification of fatty acids of the natural oil by mixing the natural oil with a lipase and ethanol, to obtain a mixture comprising LCPUFAs on glyceride form and LCMUFAs on ethyl ester form;
ii) distilling the mixture of step i) to separate LCMUFAs as one fraction and LCPUFAs as another fraction, collecting the LCMUFA fraction to obtain the LCMUFA enriched composition.

12. The method of claim 11,
for preparing an enriched LCMUFA composition comprising at least 16.0 wt. % cetoleic acid, at least 3 wt. % C20:5 n3 (EPA), any concentration of DHA a maximum of 4 wt. %, and an EPA:DHA ratio of at least 4:1,
wherein the fatty acids of the composition are derived from the natural oil comprising cetoleic acid and EPA, and
the composition comprises the fatty acids in form of ethyl esters, free fatty acids or fatty acid salts.

13. The method of claim 11, for preparing an enriched LCMUFA composition wherein the concentration of cetolic acid is at least 39%, and the concentration of EPA is at least 5.0%.

14. The method of claim 11 wherein the natural oil is derived from herring, mackerel, capelin, cod, saithe or tobis.

15. The method of claim 11 wherein the fatty acids of the natural oil are mainly on the form of triglycerides.

16. The method of claim 11 wherein the lipase is a 1,3-specific lipase.

17. The method of claim 11 wherein the lipase is selected from the group of Rhizomucor miehei lipase, Aspergillus niger lipase, Thermomyces lanuginosus lipase, Candida antarctica lipase, Candida rugosa lipase, Geotrichum candidum lipase, Penicillium roguefortii lipase, Rhizopus delemar lipase, and Rhizopus oryzae lipase.

18. Use of an LCMUFA composition according to claim 1 in food, feed, supplements or nutraceuticals.

19. The LCMUFA composition according to claim 1 for use in therapy.

20. The LCMUFA composition for use according to claim 19, for use in treatment of one or more of metabolic syndrome, insulin sensitivity, fatty liver disease (NAFLD, NASH), diabetes, prediabetes, diabetic neuropathy, raised LDL-cholesterol, cardiovascular disease and risk factors, and skin diseases or conditions.

21. The LCMUFA composition for use according to claim 19, wherein the use is in skin health or treatment of skin.

22. The LCMUFA composition for use according to claim 19, wherein the treatment of skin contributes to the skin's barrier function, keeping the skin appearing healthy, avoiding wrinkled skin or red spots, or protects against negative effects on the skin from the sun's UV radiation.

23. The LCMUFA composition for use according to claim 19, wherein the skin diseases and conditions are selected from the group of eczema, psoriasis, dermatitis, acne and rosacea (papulopustular rosacea).

24. LCMUFA composition for use according to claim 19 comprised in a pharmaceutical composition; supplement, i.e. a food, nutritional, or dietary supplement; a medical food; or food for special medical purposes.
